(19) United States
(12) Patent Application Publication
(10) Pub. No.: US 2012/0277664 A1
(43) Pub. Date: Nov. 1, 2012

(54) METHOD AND DEVICE FOR THE IRRIGATION AND DRAINAGE OF WOUNDS, TUBES, AND BODY ORIFICES

(76) Inventor: Bradford Macy, JR., Concord, CA (US)

(21) Appl. No.: 13/458,588

(22) Filed: Apr. 27, 2012

Related U.S. Application Data
(60) Provisional application No. 61/480,298, filed on Apr. 28, 2011.

(51) Int. Cl. A61M 3/02; A61M 1/00

(52) U.S. Cl. 604/28; 604/38

(57) ABSTRACT

A multi-barrel syringe is disclosed for the dual function of delivering and drawing fluid from a target site. The syringe has at least two barrels that may be positioned coaxially relative to one another. One barrel may be pre-filled with sterile delivery fluid designed to irrigate catheter tubing. Another barrel may be provided for removing delivered fluid or other body fluid from a site. The syringe may orientate the barrels such that fluid passing in one barrel does not contact the other barrel. The syringe includes collapsible and extendable plungers for each barrel where each plunger is length adjustable. While in use, the syringe includes a locking mechanism to prevent movement of other plungers while one plunger is in use. Methods of using the described syringe are also provided.

### CROSS REFERENCE TO RELATED APPLICATIONS

This application claims the benefit under 35 U.S.C. §119 of U.S. Application No. 61/480,298 filed Apr. 28, 2011, the disclosure of which is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

Described herein are various methods, systems, and devices for the irrigation of a body orifice or tube inserted therein, and more particularly, to improved aseptic methods, systems and devices for the same.

### BACKGROUND

Current wound and tube irrigation techniques put the patient, clinician and community at unnecessary risk of body fluid exposure. It has been shown that catheter associated urinary tract infections are a large contributor to the emergence and increase in antibiotic resistant bacterial infections. One reason for this is cross contamination during irrigation and drainage procedures. Cross contamination of pathogenic organisms occurs frequently in the clinical setting because state of the art technology does not address the problem appropriately. The current standard procedure for the irrigation of a urinary catheter as described below shows the serious need for improved technology to decrease cross contamination, re-infection, simplify the process, and protect patients, clinicians and the community from bio-hazardous waste exposure.

Clinicians must currently use several items to irrigate a urinary catheter. They employ an irrigation tray, a syringe, a separate bottle of irrigation solution, and a receptacle to hold irrigation solution. They have to pour the solution into a receptacle, pull the solution from the receptacle into the syringe, and then irrigate the catheter by pushing the sterile irrigation fluid through the catheter and into the bladder. The contaminated fluid is removed from the bladder into the same barrel that the sterile irrigating fluid occupied. The used fluid—contaminated with bio-hazardous waste—is squirted from the syringe into the flimsy irrigation tray. The fluid is easily splashed out of the tray during this process, or drips out of the end of the syringe, and often ends up on bed sheets, the floor, the clinician, or the patient. This flimsy tray must be carried to a toilet or other disposal site unsealed, which can result in spills or splashes. If further irrigation is needed, the same syringe that had contaminated waste is reused and the contaminated barrel is refilled with “sterile” irrigation fluid, now contaminated. After the procedure is complete, the tray and syringe is often re-used many times which can re-infect the patient, or cause cross contamination and the spread of infection within the immediate environment.

### SUMMARY OF THE DISCLOSURE

The task of irrigating and draining areas of a patient's body often requires multiple instruments with a heightened risk of contamination and infection. As such, one aspect of the described invention relates to a hygienic irrigation and drainage syringe and methods of using the described syringes for irrigating wounds, tubes, and body orifices in the medical setting.

Some embodiments provide for methods and devices for irrigating tubes and wounds in a safe, easy, and cost effective manner to reduce medical waste and prevent infection of the treatment site. Some embodiments described provide for an irrigation procedure to be performed with a single device, without the need for an irrigation tray or separate irrigation solution or receptacle.

Some embodiments provide for an irrigation and drainage syringe having a first barrel, a second barrel, where the second barrel is coaxial with the first barrel. The syringe may also include a nozzle positioned at a distal end of the syringe, wherein the nozzle comprises a tip portion that may be further comprised of a first and second tip. In some variations, the first tip is positioned at least partially within the second tip.

In further embodiments, a syringe may include multiple passageways defined by the barrels, tips, or nozzles. In some embodiments, a first passageway defined by the first barrel and first tip, wherein the first barrel is configured to store and move a first fluid in and out of the first passageway. In other embodiments, the syringe has a second passageway defined by the second barrel and second tip, wherein the second barrel is configured to store and move a second fluid in and out of the second passageway. The fluid in the first passageway may be separated from the second fluid in the second passageway such that the first fluid does not contact the second passageway. Additionally, some embodiments provide that the fluid in one passageway in one barrel or nozzle tip does not contact another passageway in another barrel or nozzle tip.

In other embodiments, a first plunger is connected to a first barrel and a second plunger is connected to the second barrel. Both plungers may be collapsible and extendable such that both can be moved to a collapsed or extended orientation independent of one another. The extended orientation is generally one where the plunger has an increased length compared to the collapsed orientation. In additional embodiments, the plungers can selectively move from a collapsed orientation to an extended orientation or vice versa.

In further embodiments, the first barrel of the syringe can be designed to deliver fluid, such as irrigation fluid, to a treatment site while the second barrel can be configured to collect drainage fluid. In some embodiments, the syringe is in fluid communication with a catheter to deliver and remove fluid from a site. The catheter may be in fluid communication with the syringe nozzle and the tips of the nozzle.

In some embodiments, the first and second barrel may include distal walls. Additionally, where a second barrel has a distal wall, the wall may include at least one opening and the at least one opening is configured to allow fluid to enter and exit a passageway in the second barrel or the nozzle.

In further embodiments, the first plunger and the second plunger may include one or more finger rings/holds on the proximal end of the syringe. In some embodiments, the finger hold for the first plunger is positioned between the finger holds of the second plunger.

In other embodiments, the first plunger of the syringe may include an extendable arm where the extendable arm includes a plurality of segments configured to extend relative to one another when the first plunger is moved to the extended orientation and to retract into one another when the first plunger is moved to the collapsed orientation. The second plunger may also include a plurality of segments configured to retract relative to one another allowing the second plunger to shorten in length.

In other embodiments, the second plunger is shortened in length as a sealing body attached to the plunger moves toward the proximal end of the syringe. In additional embodiments, the second plunger shortens in a length as a result of the sealing body of the second plunger moving toward the proximal end of the syringe.

In further embodiments, the second plunger may be retracted or shortened by applying a pushing, compressing, depressing, or distally directed force against the second plunger. This force applied to shorten the plunger may be provided after proximally moving the sealing body of the second plunger.

In further embodiments, the first and second plunger may include holds for a user to manually manipulate the plungers. In some embodiments, the holds can be finger rings. In additional variations, the finger holds or rings of first and second plunger can be configured to maintain a desired position relative to one another. In one embodiment, the finger holds or rings of the second plunger are positioned distal to the finger holds of the first plunger by shortening the length of the second plunger.

Additionally, embodiments also provide for syringes with a latching mechanism that can be present on one or more (or each) segment(s) for the plunger arms. In some embodiments, the latching mechanism comprises a track on an inner surface of each segment and a flexible body attached to an outer surface of each of the plurality of segments, the flexible body is configured to engage the track and preferentially allow movement of the segments in a direction that lengthens the arm of the first plunger and shortens the arm of the second plunger while resisting movement of the segments in an opposite direction.

In further variations, the syringe may comprise a locking mechanism, wherein the locking mechanism is configured to selectively lock either the first plunger or the second plunger and prevent movement of one plunger while the other plunger is in use, the locking mechanism comprising a mechanical connector for engaging a portion on either the first plunger or second plunger and to prevent movement of the engaged plunger.

Alternatively, the syringe may have a nozzle that includes a convex protrusion configured to engage a catheter, the convex protrusion positioned proximal of the distal end of the nozzle. The nozzle may also include a groove or recess along a circumference of the nozzle, the groove configured to engage a cap.

Additionally, the syringe may include a luer head adapter and syringe tip cap assembly comprising: a luer adapter; a cap; and a clamping mechanism with a compressible appendage biased toward a closed state, wherein compressing the appendage against the bias releases the clamp from the closed state.

Further embodiments provide methods for using the described syringes to irrigate and drain a portion of a patient's body. This method may include the steps of moving a first plunger of a multiple barrel syringe from a retracted state to an extended state by lengthening an arm of the first plunger; locking the first plunger in the extended state; depressing the first plunger to release fluid from a first barrel of the syringe into a portion of a patient's body, wherein the fluid passes through a first passageway; and collecting fluid from the portion of the patient's body into a second barrel of the syringe, wherein the first barrel is coaxial with the second barrel and the collected fluid does not contact the first barrel and the first passageway.

In some embodiments, the method may also include collapsing a second plunger from an extended state by proximally pulling an arm of the second plunger and thus pulling the attached sealing body from the proximal end towards the distal end to collect fluid from the portion of the patient's body. The second plunger can then collapse when it is pushed back in the distal direction allowing the second plunger to shorten in length. In some variations, this process allows the fingers, which are inserted in the finger hold(s) of the second plunger to be positioned distal to the thumb which is inserted in the finger hold(s) of the first plunger.

The method may also have the step of locking the second plunger to prevent movement of the second plunger while the first plunger is in use and/or locking the first plunger to prevent movement of the first plunger while the second plunger is in use. Further embodiments provide for the step of connecting the syringe to a catheter in fluid communication with the portion of the patient's body.

Additionally, some embodiments provide for an irrigation and drainage syringe comprising: a proximal end and a distal end; a first barrel extending along a longitudinal axis of the syringe, the first barrel configured to be coaxial with a second barrel, wherein the first barrel is partially positioned within the second barrel; a nozzle positioned at the distal end of the syringe, a first passageway defined by the first barrel; a second passageway defined by the second barrel, wherein the second barrel can hold substantially the same or greater fluid volume as the first barrel; a first plunger connected to the first barrel, wherein the first plunger is configured to move fluid into or out of the first passageway of the syringe; a second plunger connected to the second barrel, wherein the second plunger is configured to move fluid into or out of the second passageway of the syringe, wherein fluid in the second passageway does not contact the first passageway; a locking mechanism configured to selectively lock either the first plunger or the second plunger to prevent movement of the other plunger while one plunger is in use; and a latching mechanism configured to releasably maintain a length of the first plunger, wherein the length of first plunger is adjustable.

Some embodiments also provide for a syringe with two barrels allowing for sterile irrigation solution to be kept separate from the contaminated drainage which is collected in a separate barrel. The variations have a “barrel within barrel” design which saves space and improves ease of use.

Other embodiments provide for a built in receptacle to collect used, contaminated fluids, which decreases the potential for spills or splashes which cause cross contamination of bio-hazardous material during an irrigation procedure.

Further variations provide for a syringe tip that decreases the chance of fluid squirting out between the syringe tip and the catheter and spraying the clinician or environment when pressure is applied. A splash guard can be included on the nozzle portion of the syringe to protect the user if a fluid squirt does occur.

In other embodiments, a syringe tip cap is included to provide a seal which keeps contaminated fluid from dripping from the syringe after use and prior to being discarded.

Further embodiments provide for a luer adapter which allows the syringe to be adapted for use with devices demanding luer locks such as needles, cannulas, and certain types of tubing, all of which are currently employed in the medical setting for wound and body orifice irrigation.

Additionally, some embodiments provide for mechanisms for locking, extending, and retracting the arms of the syringe plungers at certain points in the irrigation and drainage procedure to allow the user to easily manipulate the syringe with thumb and fingers of one hand to both irrigate and drain a wound, tube, or body orifice easily.

### BRIEF DESCRIPTION OF THE DRAWINGS

The novel features of the invention are set forth with particularity in the claims that follow. A better understanding of various features and advantages of the embodiments described herein may be obtained by reference to the following detailed description that sets forth illustrative examples and the accompanying drawings of which:

FIG. 1A is a perspective view of an irrigation and drainage syringe filled with a delivery fluid according to one embodiment.

FIG. 1B shows the syringe of FIG. 1A with the irrigation plunger in an extended state according to one embodiment.

FIG. 1C shows the syringe of FIGS. 1A-1B filled with drainage fluid and having the drainage plunger in a collapsed state.

FIG. 2 is a perspective view of an irrigation and drainage syringe according to another embodiment.

FIG. 3A is an exploded view of the irrigation plunger arm and latching mechanism according to one embodiment.

FIG. 3B is a lateral view of an alternative irrigation plunger arm.

FIG. 4 is an exploded view of the drainage plunger arm and latching mechanism according to one embodiment.

FIG. 5 is a perspective view of a proximal portion of the irrigation and drainage syringe showing the details of a locking mechanism according to one embodiment.

FIG. 6 is an illustration of a luer head adapter and cap assembly according to one embodiment.

### DETAILED DESCRIPTION

As discussed above, treatment sites such as urinary catheter wound sites conventionally require the use of several instruments to maintain the function and cleanliness of the areas. For example, when a patient's urinary catheter is plugged; saline solution may be used to flush out and clear the tubing. In such cases, multiple instruments are currently needed to clear the catheter and drain the contaminated irrigation fluid from the treatment site. Accordingly, one aspect of the invention provides for a single device configured to hygienically irrigate and drain such a treatment site.

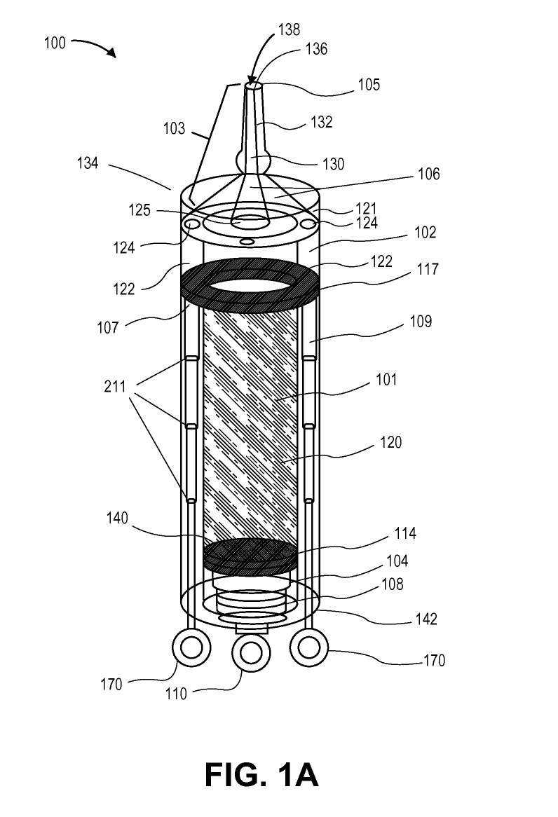

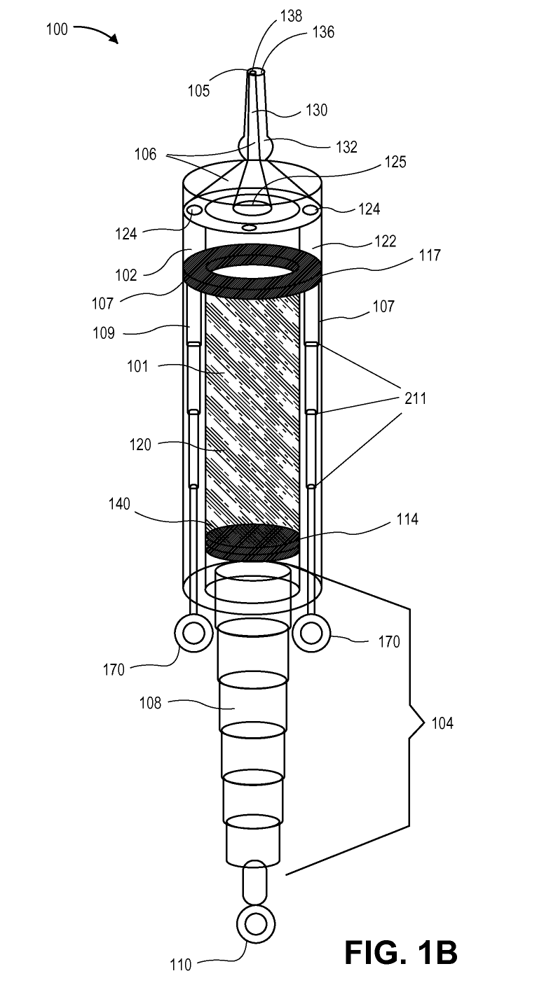

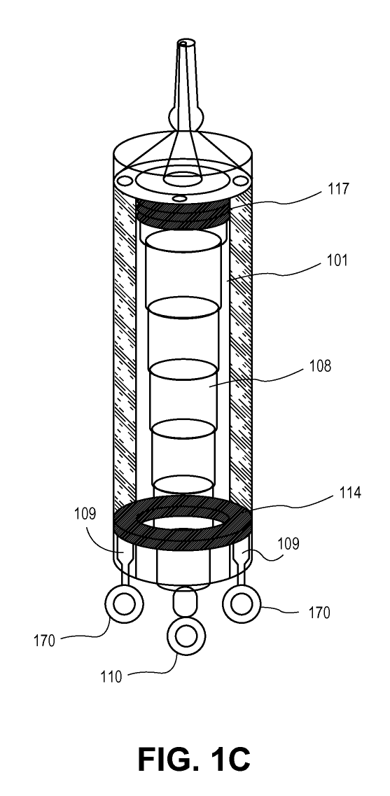

FIGS. 1A-1C show an example of one embodiment of such a device where the device is a multi-barrel syringe 100 with two compartments 120, 122, two barrels 101, 102, a nozzle portion 103, a tip portion 105, a first extendable/collapsible plunger 104, a second extendable/collapsible plunger 107, and finger rings/holds 110, 170. As shown in FIGS. 1A-1C, the first compartment 120 is defined by a first barrel 101 of syringe 100. Similarly, second compartment 122 is defined by a second barrel 102. In some variations, second compartment 122 can be defined by the area/volume in the second barrel 102 not occupied by the first barrel 101. For example, as shown in FIGS. 1A-1C, the second barrel 102 can house the first barrel 101 and the second compartment 122 can be defined by an area between the first and second barrel. In some cases, the second compartment is defined by the area between an outer surface of the first barrel 101 and an inner surface of the second barrel 122. FIG. 1C shows the filled second compartment 122 where the filled second compartment 122 is defined by the area between the first barrel 101 and the second barrel 102.

In some embodiments, the first barrel 101 can store and provide a sterile irrigation fluid to a treatment site while the second barrel 102 can drain and store fluid from the treatment site. In such cases, the first compartment 120 can be filled with a sterile fluid for delivery to a target site directly or indirectly through catheter tubing. Alternatively, the first compartment 120 can also be pre-filled with the delivery fluid prior to use. Additionally, compartment 122 can be used to remove and store the delivered fluid from the target site.

In FIGS. 1A-1C, the first compartment 120 is shown positioned within compartment 122. As such, the compartments can share a longitudinal axis and/or may be coaxial or concentrically positioned relative to one another. Other orientations also include side-by-side or tandem positioning of the compartments. Additionally, the syringe device may include more than two compartments. In some embodiments, additional barrels may provide additional compartments; however, compartments may also be formed by other syringe components such as the syringe housing.

In operation, the compartments or barrels of some embodiments can move or store substantially similar amounts of volume. In some cases, the drainage compartment or barrel must be able to accommodate at least as much volume as the irrigation compartment or barrel. For example, the irrigation compartment may store a volume of irrigation fluid destined for irrigation at a target site. Once delivered, the same contaminated irrigation fluid is drained from the target site by drawing the used fluid into the drainage compartment. To ensure that all the contaminated irrigation fluid can be stored in the drainage compartment, the volume capacity of the drainage compartment must be at least as large as the irrigation compartment.

Syringe 100, as shown in FIG. 1A, has a first barrel 101 with compartment 120 that is filled with delivery fluid. Once the delivery fluid is delivered to the target treatment site, the second barrel 102 and second compartment 122 is used to remove and store the delivered fluid from the site. FIG. 1C shows the same syringe 100 with the contaminated delivery in the second compartment 122 and barrel 102. Advantageously, the volume capacity of the second compartment 122 is at least as large as the first compartment 120. This allows substantially all of the contaminated fluid to be removed from the treatment site, which helps prevent infection. In some embodiments, the drainage compartment may have a larger volume capacity compared to the irrigation compartment to accommodate for drawing and storing the contaminated irrigation fluid as well as body fluid that may need to be drained from the site. For instance, draining fluid from a urinary catheter may include removing contaminated irrigation fluid as well as urine from the patient's bladder.

In addition to multiple compartments 120, 122 and barrels 101, 102 described, the syringe device 100 of FIGS. 1A-1C may also include a nozzle 103. In some embodiments, the nozzle 103 is positioned at a distal end 134 of the syringe 100. Both barrels may connect to the nozzle 103. The nozzle 103 may include an orifice portion 106 and a tip portion 105. The orifice portion 106 may include one or more openings, ports, outlets, or inlets for the movement of fluid into and out of the syringe 100. More particularly, the orifice portion 106 may include separate ports for controlled movement of fluid into specific barrels or compartments of the syringe 100 where a given fluid is selectively moved into one compartment or barrel without entering another compartment or barrel. For example, the orifice portion may include a first tip 130 and a second tip 132. First tip 130 may be connected to first barrel 101 such that the first tip 130 and the first barrel 101 define a first passageway extending from the port 136 to a proximal end 140 of the first barrel 101. Similarly, the second tip 132 may be connected to the second barrel 102 and define a second passageway extending from the port 138 through the second barrel 102 and to the proximal end 142 of the syringe 100. In some embodiments, the first barrel 101 may be housed within the second barrel 102 and the second passageway is defined by an outside surface of the first barrel 101, an inside surface of the second barrel 102, and the second tip 132.

In some embodiments, the movement and storage of fluid through each barrel or compartment is substantially confined to the passageway defined by the specific barrel or compartment. For example, movement of irrigation fluid through first barrel 120 is substantially confined to the passageway defined by the first tip 130 and first barrel 101. Likewise, movement of drainage fluid into compartment 122 is substantially confined to movement of the fluid into the passageway defined by the second tip 132 and the second barrel 102. Advantageously, in the contemplated embodiments, the fluid from one barrel does not contact the passageway defined by another barrel. As such, the character of the fluid is maintained during movement into and out of the device. For example, sterile fluid in one passageway does not contact the surface of another passageway, which avoids possible contamination.

In further embodiments, the outlets 136, 138 of the first and second tips 130, 132 are located at a tip portion 105. The distance between the outlets 136, 138 and the distal end 134 of the syringe 100 can vary. In some embodiments, the lengths of the first and second tips are selected such that the outlets 136, 138 are positioned at the same distance from a distal end 134 of the syringe. In such cases, the first tip 130 may be housed partially or wholly within the second tip 132. In other embodiments, the outlets 136, 138 are positioned a varying distances from the distal end 134 of the syringe and the first and second tip may partially but not completely overlap.

In other variations, the orifice portion 106 of nozzle 103 may be connected with opening leading directly into the first and second barrels. As shown in FIGS. 1A-1C, first tip 130 leads into opening 125 in the first barrel and second tip 130 leads into openings 124 into the second barrel. The openings 124, 125 allow fluid flow from the first and second tip into respective barrels.

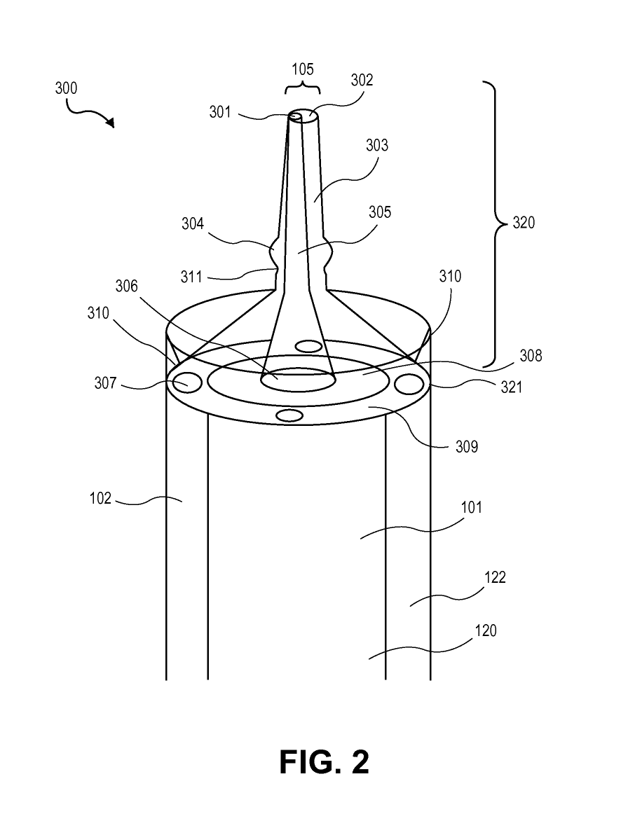

FIG. 2 provides an alternative embodiment of a multi-barrel irrigation and drainage syringe with a modified nozzle and tip portion. FIG. 2 shows an irrigation and drainage syringe 300 having a first barrel 101, a second barrel 102, a first compartment 120, a second compartment 122, and a nozzle 320. The nozzle 320 has a tip portion 105 with two separate openings 301, 302 which connect with two separate orifices 305, 303 respectively. These orifices 305, 303 lead to openings 306, 307 which lead into the barrels 101 and 102 respectively.

As shown in FIG. 2, in some embodiments, the irrigation barrel 101 may have a distal wall 308 that engages a distal wall 309 of the drainage barrel 102. In some variations, the distal wall 308 and distal wall 309 of the barrels are flush with one another. In other embodiments, the walls 308, 309 may be offset from one another. In further variations, the distal wall 309 of the second barrel 102 may be part of the first barrel 101 such as an extension of the distal wall 308. For example, the distal wall 309 of the second barrel 102 may be a flange, rim, or lip of the first barrel 101. In other embodiments, a sealing body (not shown) may be placed between the distal wall 308 of the first barrel 101 and the distal wall 309 of the second barrel 102 to create a tight seal between the walls 308, 309 to prevent leakage of gas or fluid between the barrels 101, 102. Additionally, the distal wall 308 may include openings, ports, outlets, or inlets 306 to allow passage of fluid or gas through the distal wall 308 into the first barrel 101 (shown as opening 125 in FIG. 1A). Likewise, distal wall 309 of the second barrel 102 may include openings, ports, outlets, or inlets 307 (also shown as openings 124 in FIGS. 1A-1C) to allow passage of fluid or gas through the distal wall 309 into the second barrel 102.

Additionally, open hollow passageways can also be formed by the orifices, openings, and tips described. For example, a passageway can lead from the opening 301 in the tip 105 to the opening or openings 306 in the distal wall 308 of the irrigation barrel 101. The passageway may be made from a hollow cylindrical shaped plastic body which could be attached to the nozzle 320 or tip 105 at one point and attached to a distal wall 308 of the irrigation barrel 308 at another point. The passageway allows fluid or gas to move through the barrel opening 306 and tip opening 301 when the first plunger is depressed or pushed. Additionally, a passageway can lead from the opening 302 in the nozzle 320 and tip 105 to the opening or openings 307 in the distal wall 309 of the drainage barrel 102. This passageway can accommodate movement of fluid or gas movement through opening 302 and openings 307 into and out of the second barrel 102 when the second plunger 107 is pulled proximally.

In some embodiments, the nozzle 320 is designed to fit into a urinary catheter or other similar tubing. In such embodiments, the nozzle 320 has a protrusion or projection 304 configured to engage and releasably hold an end of a catheter or other tubing. FIG. 2 shows the nozzle 320 with a protrusion 304 extending 360 degrees around the nozzle 320. In other embodiments, the protrusion 304 extends around a portion of the nozzle 320 but not completely around the nozzle. The protrusion 304 can be located anywhere along the nozzle. In some embodiments, the protrusion 304 is located proximal to the tip 105. The protrusion 304 can be placed one-third to three-quarters of the distance between the tip 105 and a distal wall of the first or second barrel 101, 102. In other embodiments, the protrusion 304 is one-third to three-quarters of the way down from the tip 105. In further variations, the protrusion 304 may be closer to the tip 105 than to the distal end of the syringe 300 or the base 321 of the nozzle 320. For example, the protrusion 304 may be positioned distal of the distal end of the syringe 300 but proximal of the tip 105 such that the protrusion 304 is positioned at a point that is approximately one-third the distance between the distal end of the syringe 300 and the tip 105.

In some variations, the protrusion 304 may have a convex shape. This convexity can provide for a tighter hold of a latex, silicone or any other soft plastic tube by stretching the tube over the convexity. This stretching allows for a firmer hold of the catheter or tube, protecting it from coming off or squirting fluid during the irrigation or aspiration procedure.

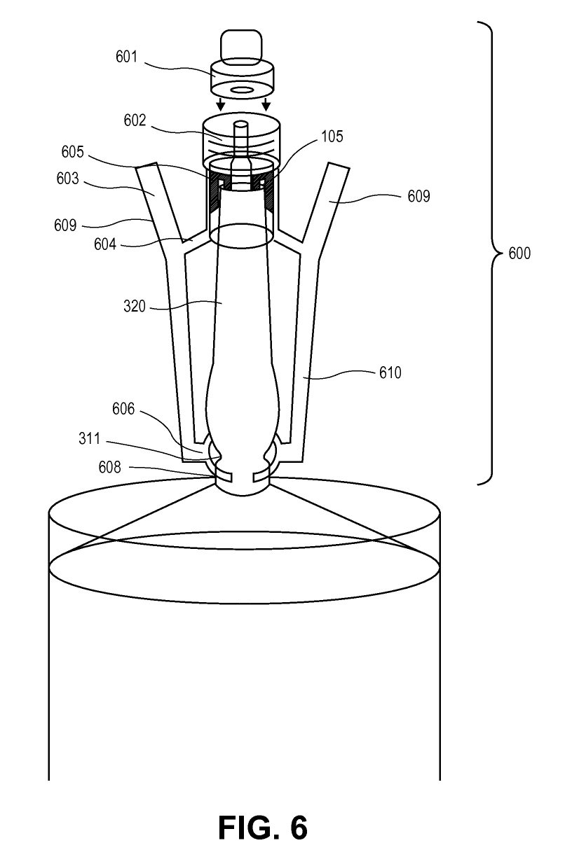

Additionally, the nozzle 320 of FIG. 2 also includes a cap holding portion 311. A cap holder 311 may be a recessed portion or indentation on the nozzle 320 designed for receiving a cap or luer connector. In some variations, the recess spans 360 degrees around a circumference of the nozzle 320. The cap holder may be placed distal or proximal relative to the protrusion 304. In some embodiments, the cap holder is distal to a convex protrusion 304. In some variations, the cap holder 311 can hold a luer head adapter and syringe tip cap 600 which is described herein (see FIG. 6).

The embodiments contemplated may also include a splash guard. The splash guard catches fluid sprayed out from the interface of the syringe tip 105 or nozzle 320 with a tube, protecting the user from being splashed during a procedure. As shown in FIG. 2, the splash guard 310 can be placed at the base 321 of the nozzle 320. The splash guard 310 is a 360 degree continuation of the outer wall of the drainage barrel 122 and extends beyond the distal wall 309 of the drainage barrel 102. In some variations, the splash guard extends around the syringe 300 but does not span 360 degrees. In other embodiments, the splash guard 310 continues the length of the outer wall of the second barrel 122 by approximately ½″ to 1″. However, any suitable amount of length for the splash guard 310 can be used. FIGS. 1A-1C show a similar splash guard 121 that can also extend beyond the second barrel 102.

In addition to the above described features, some embodiments can include one or more plunders. As shown in FIGS. 1A-1C, syringe 100 may include a first plunger 104 housed within the first barrel 101. In some embodiments, the first plunger 104 includes a sealing member 114, such as a gasket, at a distal end of the first plunger 104 to provide an effective seal between the plunger and the first barrel 101.

In further embodiments, the first plunger 104 has an extendable plunger arm 108 at a proximal end 142 of the syringe 100. In such embodiments, the first plunger 104 can be reversibly and releasably adjusted from a collapsed state to an extended state by, for example, lengthening the arm 108 of the first plunger 104. In FIG. 1A, the plunger arm 108 is locked in a collapsed state where the length of the arm 108 is in a shortened position. The shortened plunger arm 108 may be preferred in some instances such as for shipment to allow compact packaging in transit. When the unit is unpackaged and prepared for use, the plunger arm 108 may be extended from the collapsed to an extended position by lengthening the arm 108 and locking the arm 108 in the extended orientation. FIG. 1B shows an embodiment of the syringe 100 where the first plunger 104 is in the extended orientation and the arm 108 has been lengthened for use. Once lengthened, a user can depress the first plunger 104 in a distal direction to deliver fluid in barrel 101 through tip 130.

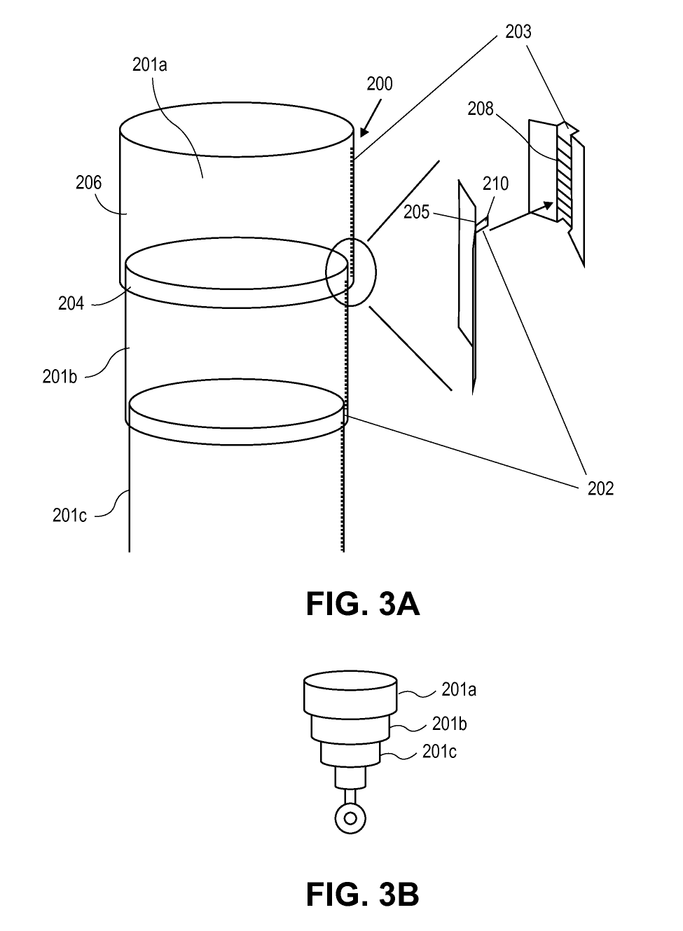

To allow length adjustment, some embodiments provide a plunger arm 108 with subunits designed to move relative to one another. For example, as shown in FIG. 3A, a plunger arm 108 may have several cylindrical subunits or segments 201a-c which can interconnect with each other. One or more of the subunits 201a-c may include a latching mechanism 200 to releasably maintain the arm 108 at a desired length. In some embodiments, the latching mechanism 200 includes a flexible body 202 that protrudes or projects from a surface of a subunit 201b. The flexible body 202 may project at an angle between 0-180 degrees. As shown in FIG. 3A, the flexible body 202 projects at approximately 45 degrees from the outer surface 204 of the subunit 201b. In some embodiments, the flexible body 202 is angled such that the tip 210 of the flexible body 202 is distal to the base 205 of the flexible body 202. In other embodiments, the flexible body 202 of the plunger arm 108 is angled at approximately 45 degrees facing the tip portion of the syringe.

The flexible body 202 can be designed to interface with a track 203 on another surface 206, such as an inner surface, of an adjacent subunit 201a. The track 203 may be a recessed portion of the subunit 201a with multiple grooves or indentations 208 extending along a surface of the subunit 201a. In some embodiments, the track 203 has multiple gashes 208 on the inside surface 206 of the recessed portion which run vertically from the top of the subunit 201a to the bottom. In other embodiments, the track 203 may only run partially along a top to bottom surface of the subunit 201a.

To extend and maintain the length of a plunger arm 108, the grooves 208 interface with the flexible body 202. In some embodiments, the flexible body 202 is designed to allow the flexible body 202 to move across the grooves 208 in one direction but to resist movement in another direction. For example, in FIG. 3A, the flexible body 202 is positioned to allow bending over the grooves 208 when moved proximally along the longitudinal axis of the arm 108, but to oppose bending in the distal direction. In some embodiments, the surfaces of the subunits may partially overlap in the extended orientation in order to allow a flexible body 202 positioned on the outer surface 204 of one subunit 201b to come into contact with an inner surface 206 of an adjacent subunit 201a and lock the arm 108 in the extended orientation.

In operation, the plunger arm 108 may be collapsed prior to delivery of fluid. Once the first plunger 104 is needed, the user may proximally extend the arm 108 by lengthening the arm 108 in the proximal direction. In some embodiments, the subunits telescopically move relative to one another. Once the subunits are moved to an extended orientation, the latching mechanism shown in FIG. 3A maintains the length of the arm 108 and the position of the subunits 201a-c relative to each other. In some embodiments, the latching mechanism may not be needed on each subunit, but rather on at least one subunit. In other embodiments, the arm 108 can be released from the latching mechanism and the subunits 201a-c can be retracted into one another to the collapsed or retreated orientation. Although shown in FIG. 3A has having relatively the same shape and size, the subunits 201a-c may be of different dimensions. For example, FIG. 3B shows subunits having varying cross-sections. Moreover, in other embodiments, the latching mechanism may not require overlap of surfaces such as where the aim is extendable by screwing on extension units to the arm where the extension units are matingly connected to the arm. The mating connection may be a male and female threaded connection where the extension unit can thread onto a receiving portion on the plunger arm 108.

In addition to an extendable first plunger 104, the syringe 100 may also include a collapsible and extendable second plunger 107. In some embodiments, the second plunger 107 is within the second barrel 102 and has a plunger arm 109 attached which extends out of the second barrel 102 at the proximal end 142 of the syringe 100.

Second plunger 107 may include one or more plunger arms 109 for drawing fluid into the second barrel 102. The second plunger 109 may also include a sealing body 117 connected to the plunger arms 109. The sealing body may be positioned in compartment 122 within the passageway defined by the second barrel 102 and second tip 132. In some embodiments, the sealing body 117 includes an opening to accommodate the first barrel 101 through the opening. In such cases, the sealing body 117 provides a seal between an outer surface of the first barrel 101, the second plunger 109, and an inner surface of the second barrel 102 such that fluid in the second compartment 122 does not leak out of the second barrel 102.

In some embodiments the second plunger 107 has a collapsible and extendable plunger arm 109 where the length of the arm 109 can be increased or decreased adjustably. As shown in FIG. 1A, the arms 109 are in an extended increased length orientation. In the extended orientation, the plunger arms 109 are housed partially in the second barrel 102 with a portion of the arms 109 near the proximal end of the syringe. The portion of the arms 109 at the proximal end 142 can be pulled by the user to move the sealing body 117 proximally within the second barrel 102. Once pulled, the sealing body 117 creates a vacuum in the second compartment 122 that results in drawing gas or fluid into the compartment 122. As the plunger arm 109 and the sealing body 117 are moved proximally, a greater portion of the plunger arms 109 can be positioned outside the second barrel 102.

To collapse the plunger 107, the length of the arms 109 is shortened. In some embodiments, the arms 109 are made of a plurality subunits 211 that are designed to retract into one another to shorten the length of the arms 109. The subunits 211 may be multiple segments that move relative to one another and can retract into one another in one direction. FIG. 1C shows the collapsed second plunger 107 with retracted arms 109.

Alternatively, as the plunger arm(s) 109 and the sealing body 117 are moved proximally, the plunger arms 109 may be retracted during the proximal movement. This may be accomplished by applying a distally directed force such as by way of depression, compression, or pushing. The distally directed force allows the plunger arm(s) 109 to retract to a shortened length and collapsed state. The length of the plunger arm(s) 109 can be shortened by varying degrees from just slightly shorter than the maximum length to the shortest length possible for the arm(s) 109.

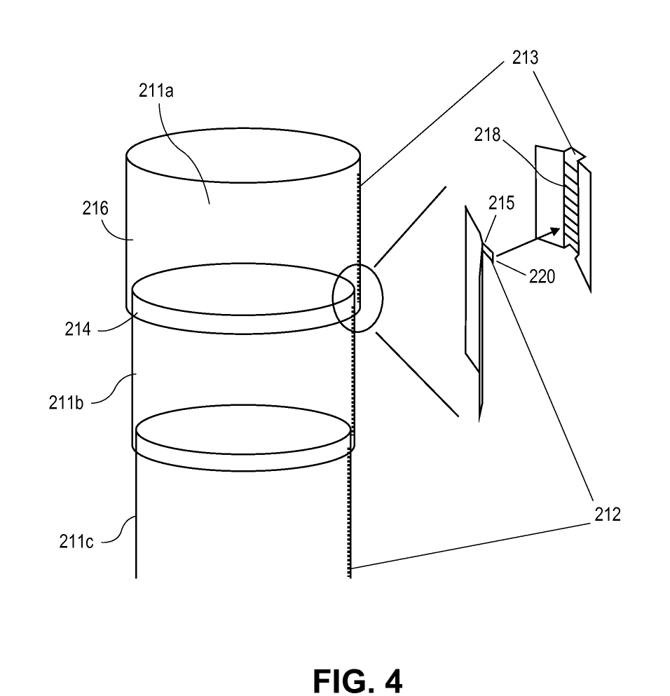

Some embodiments include a latching mechanism for maintaining the length of the plunger 107. For example, as shown in FIG. 4, a latching mechanism may include a flexible body 212 attached to outer surface 214 of a subunit 211b and a track 213 attached to an inner surface 216 of an adjacent subunit 211a. The flexible body 212 may project at an angle between 0-180 degrees. As shown in FIG. 4, the flexible body 212 projects at approximately 45 degrees from the outer surface 214 of the subunit 211b. In some embodiments, the flexible body 202 is angled such that the tip 220 of the flexible body 212 is proximal to the base 215 of the flexible body 212. In other embodiments, the flexible body 212 of the plunger arms 109 is angled at approximately 45 degrees facing the user direction of the syringe.

The flexible body 212 can be designed to interface with a track 213 on another surface 216, such as an inner surface, of an adjacent subunit 211a. The track 213 may be a recessed portion of the subunit 211a with multiple grooves or indentations 218 extending along a surface of the subunit 211a. In some embodiments, the track 213 has multiple gashes 218 on the inside surface 216 of the recessed portion which run vertically from the top of the subunit 211a to the bottom. In other embodiments, the track 213 may only run partially along a top to bottom surface of the subunit 211a.

Further embodiments provide for irrigation and drainage syringes with finger rings/holds attached to the plungers. As shown in FIG. 1A and 1B, both plunger arms 108, 109 may have finger rings 110, 170 to insert the fingers and thumb to allow easy pushing and pulling action on the plungers 102, 104. In some variations, the syringe 100 can have a first irrigation barrel 101 with a plunger arm 108 with one finger ring 110 and a second drainage barrel with more than one plunger arm 109 and more than one finger ring 170. In some embodiments, the plunger for the first barrel or irrigation barrel is centered between the plungers for the drainage barrel. This allows the user to place the thumb in the irrigation plunger ring, and the forefinger and middle finger in the drainage plunger rings, which provides for comfortable one handed use of the device.

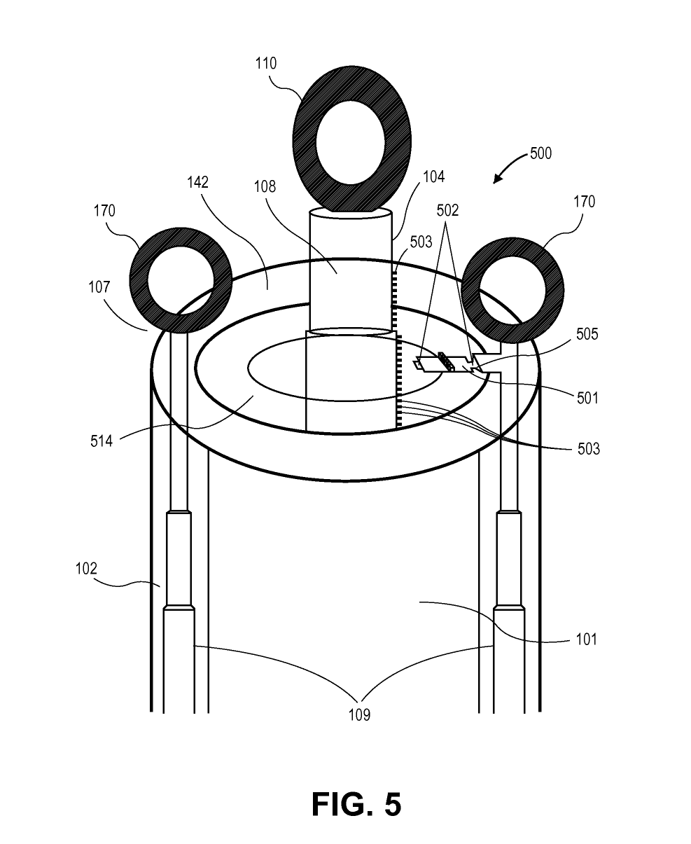

In some embodiments, as shown in FIG. 5, a locking mechanism 500 can be used to selectively lock the first plunger 104 or the second plunger 107, to keep one from moving while the other one is in use. The locking mechanism 500 can be located on the proximal end 142 of the syringe such as on the proximal wall 514 of the first barrel 101. In some embodiments, the locking mechanism is a mechanical fit or mated lock. For example, a lock 501 can be attached to the proximal wall 514 and moved back and forth between the first and second plungers 104, 107, locking either the first or second plunger 104,107. The lock 501 may have small latches 502 on two opposing sides. On one side the latches 502 slide into openings or gashes 503 which run vertically along one area of the outer surface of the first plunger arm 108. On the other side the latches 502 slide into a protrusion or projection 505 located on the plunger arm 109. In some embodiments, the projection 505 is located between the finger ring 170 of plunger arm 109 and the proximal end 142 of the syringe. In other embodiments, the latches slide into a protruding body 505 which is located just proximal to the finger ring 170 on the drainage plunger aim 109.

When the lock 501 is pushed toward the first plunger arm 108, the latch engages with one of the grooves 503 in the first plunger arm 108 and locks it from movement allowing only the second plunger 107 to move. By immobilizing the first plunger 104 as such, it provides leverage for the user to proximally pull on the second plunger 107 to draw gas or fluid into the second compartment and second barrel of the syringe. This can be accomplished by having one digit (usually the thumb) in the finger ring 110 on the first plunger arm 108, and the forefinger and middle finger in the two second plunger arm 109 finger rings 170.

Alternatively, when the lock 501 is pushed toward the protruding body 505 on the second plunger arm 109, the latches 502 engage the protruding body 505 such that when the first plunger 104 is in use, the second plunger cannot be moved. By immobilizing the second plunger arms 109 as such, it allows the user to push on the first plunger 104 with the thumb while the fingers are inserted in the second plunger 107 finger holes 170 for leverage.

In some embodiments, the placement of the locking mechanism allows the first and second plungers to be locked in the collapsed state with a shortened or the shortest length. For example, the first plunger 104 can be locked in the collapsed orientation where the plunger 104 is at its shortest length and the plunger arm 108 has not been expanded or extended. Similarly, the second plunger 107 can be shortened after being proximally pulled out of the second barrel 102 by retracting the arms 109. As the arms 109 retract, the protrusion 505 can then be moved toward the lock 501 such that the second plunger 107 can be locked in a shortened or shortest length where the aims 109 are partially or fully retracted. Additionally, in other embodiments, more than one locking mechanism or locking protrusion may be used if multiple plungers or multiple plunger arms need to be locked during use.

FIG. 6 is an illustration of a luer head adapter and syringe tip cap assembly 600 that can be used with the described syringes. In this embodiment, the assembly 600 seals the syringe, keeping fluid within the device from leaking out and maintaining a sterile seal. The assembly also serves to adapt the syringe for use with luer locking devices. In some embodiments, the assembly 600 has a twist off cap 601, a luer adapter 602, and a clamping mechanism. The cap 601 may be made of soft plastic to provide a seal for the assembly 600. Twisting the cap 601 breaks the seal for use. In other embodiments, other types of seals may be used. The cap 601 can releasably twist onto and off the assembly after use to keep fluids from dripping out of the syringe prior to disposal.

The assembly 600 may also include a luer adapter 602 that can attach to any needle, tube, or other device designed to interface with a luer lock. A distal end of the luer adapter can releasably engage the cap 601 to allow access to the syringe tip. Releasable engagement may be accommodated by mated connection between the cap 601 and the adapter 602 such as a threaded path on a surface of the cap 601 and adapter 602 allowing the cap to twist onto the adapter. When the cap 601 is not engaged, any compatible tubing, needle, etc. may be attached to the luer adapter to connect the syringe tip to the tubing, needle, etc. A proximal end of the luer adapter 602 can engage the tip 105 of the nozzle 320. The proximal end of the luer adapter may be configured to slide over the tip 105. The syringe tip 105 can fit snugly into the luer adapter 602 by way of syringe tip seal 605, which seals the syringe tip 105 to the luer adapter 602 and keeps the device from leaking during storage and use. This seal may be made of rubber, silicone or soft plastic. In some embodiments this seal may be a part of the syringe tip 105. In some embodiments, the assembly 600 may be used with a nozzle 320 with a protrusion 304. Alternatively, the assembly 600 with a luer adapter 602 may be used without a protrusion 304 as the luer adapter 602 may be designed to interface with tubing and hold onto tubing during procedures.

In some embodiments, the luer head adapter and syringe tip cap assembly 600 is held in place onto the tip 105 by a clamping mechanism. This mechanism has two upper appendages 603 which, when squeezed, usually by the forefinger and thumb of the hand, bend two middle appendages 604 causing two lower appendages 606 to spread apart, disconnecting them from the syringe tip. The clamping mechanism may have open and closed states where the appendages may be biased toward a closed state. By compressing on one or more of the appendages, the clamping mechanism can be opened to receive the syringe tip 105 or release the tip 105. The assembly 600 can then be pulled off and the syringe used. The assembly 600 can be replaced or re-clamped into place after the syringe is used to keep fluid from leaking out of the syringe prior to discarding.

In some embodiments, the clamping mechanism may include compression tabs 603, struts 604, and a base 608. The compression tabs may equidistant from a longitudinal axis of the assembly 600 along the length of the tabs. In other embodiments, some portions of the compression tabs 603 are at a first distance from the longitudinal axis of the assembly 600 and other portions of the tabs 603 are at a second, different distance. For example, in FIG. 6, first portion 610 is at a shorter distance from the longitudinal axis of the assembly 600 compared to a second portion 609. In other embodiments, the distance between the compression tabs 603 and the longitudinal axis of the assembly 600 gradually decreases from a distal to proximal end of the assembly 600, resulting in a tapering effect. The clamping mechanism can also include struts 604 that provide additional stability to the clamping mechanism. Struts 604 can also serve as pivot point, hinge, or fulcrum for moving a portion of the compression tabs 603 outward from the longitudinal axis of the assembly 600. In some embodiments, a first portion 610 of the compression tabs 603 may be biased inward toward the longitudinal axis of the assembly 600 for a closed state. To open the clamping mechanism, force is applied to another portion 609 to pivot the first portion 610 against the struts 604 and release the syringe tip from the clamp.

In further embodiments, the base 608 of the clamping mechanism is shaped to accommodate the shape of the syringe nozzle 320. Where a syringe includes a cap holding portion 311 (as described above), the base 608 may be designed to accommodate a recessed portion or indentation 311 in the nozzle 320.

Another aspect of the embodiments described is a method for irrigating and draining a treatment site. For example, a clinician may use the described multi-barrel syringe in FIGS. 1A-1C to irrigate and drain a urinary catheter. The clinician may first fill the first barrel 101 with irrigation fluid (alternatively, the barrel 101 may come pre-filled with irrigation fluid) such as saline. The syringe 100 is then connected to an end of a urinary catheter. To connect the catheter, the syringe 100 may be attached by a luer head adapter and syringe tip cap assembly 600 or by tightly fitting the catheter over the nozzle 103, 320. Connecting the tip of the syringe to the catheter may also entail fitting the catheter over a protrusion on the nozzle 103,320.

Once connected, the first plunger 104 of the first barrel 101 may be moved from a collapsed or retracted state to an expanded or extended orientation. This can be done by increasing the length of an arm 108 of first plunger 104. The first plunger can be lengthened for example by moving subunits 201a-c in the arm 108 relative to one another. Once lengthened, the arm 108 can be latched or locked in the lengthened orientation. The user can push or press the plunger 104 to deliver the irrigation fluid through the first barrel 101 into the catheter. By distally moving the sealing body 114, the plunger 104 distally pushes the irrigation fluid out of compartment 120. The irrigation fluid moves through the first barrel 101, first tip 130 or orifice 305, and out through the tip portion 105 via opening 136 or opening 301.

In moving the irrigation fluid out of compartment 120 and the first barrel 101, the irrigation fluid does not contact the second barrel 102 or a passageway defined by the second barrel 102 or a passageway defined by the nozzle 320 and opening 302. In some variations, the first plunger has a finger ring 110. A digit such as a thumb or finger can be placed into the ring and used to manipulate the plunger 104 by pulling, pushing, depressing, compressing, or otherwise applying force to the plunger.

In other variations, prior to delivery, a drainage or second plunger 107 is locked into place to prevent movement of the drainage plunger 107 while the first plunger 104 is in use. Locking the second plunger 107 allows the user to manipulate the first plunger 104 without accidentally drawing fluid into the second barrel 102. Additionally, where a second plunger 107 has finger rings 170, the user can place fingers in the finger rings 170 to assist in manipulating the first plunger 104. For example, the locked second plunger 107 may have finger rings 170 positioned distal to the finger ring 110 of the first plunger 104 when the plunger arm 108 is extended. The user may place his index and middle finger in the finger rings of the second plunger while placing his thumb in the finger ring 110 of the first plunger 104. While pushing the first plunger 108 to deliver fluid, the user may use the finger rings 170 of the locked second plunger 107 for leverage.

Once the irrigation fluid is delivered, the syringe can be used to remove fluid from the treatment site by drawing fluid into the second barrel 102. To draw and collect fluid, the second plunger 107 is manipulated to pull fluid through opening 138 (or opening 303 in an alternative embodiment). As described, the second plunger 107 may have one or more finger holds or rings 170 to accommodate a digit such as a thumb or finger. A thumb and/or finger(s) can be placed into the rings 170 and used to manipulate the plunger 107 by pulling, pushing, depressing, compressing, or otherwise applying force to the plunger 107.

In some embodiments, the second plunger 107 has a sealing body 117 that is connected to arms 109 of the plunger 107 such that when force is applied to the second plunger 107, the sealing body 117 is proximally moved. When the sealing body 117 is moved proximally, a vacuum can be created in the second barrel 102 that pulls or draws fluid from a target site into the second barrel 102. The sealing body may be proximally moved by proximally pulling on the plunger 107.

When drawn into the second barrel, the collected fluid flows into opening 138 or opening 302 , through second tip 132 or orifice 303, through openings 124 or openings 307, and into the second compartment 122 of the second barrel 102. Depending on the volume of fluid collected, the sealing body 117 may be proximally moved part of the way or completely toward the proximal end 142 of the syringe 100. Moreover, the fluid collected into the second barrel flows to a passageway defined at least by the second barrel 102 and the opening 138 or opening 302. Furthermore, in some embodiments, the fluid collected in the second barrel flows only through the passageway defined by the second barrel 102 and the opening 138, 302 but does not contact a passageway within the first barrel 101, first tip 130, or orifice 305.

In further variations, the second plunger 107 may include collapsible, retractable, and extendable arms that allow the length of the plunger 107 to be adjusted. In such cases, the second plunger 107 may be in an extended state initially prior to collecting fluid. As shown in FIG. 1A and 1B, initially, the second plunger 107 may be housed substantially within the second barrel 102. However, during use, the second plunger 107 may be proximally pulled to move the sealing body 117 proximally. This may result in a portion of the second plunger 107 moving outside the second barrel 102. In further variations, the first plunger may be locked while the second plunger is used to prevent the first plunger from moving while moving the second plunger.

To reduce the portion of the second plunger 107 present outside the second barrel 102, the length of the second plunger 107 can be shortened by retracting the arms 109 of the plunger 107. The arms 109 may be made of a plurality of subunits 211 that can retract into one another to shorten length as described above. In some embodiments, the arms 109 are retracted by applying a distally directed force against the arms 109 that results in retracting the subunits 211 into one another. FIG. 1C shows the retracted arms 109 of the plunger 107 after the second barrel 102 has been filled with collected fluid.

As described, the first and second plungers 104, 107 may include finger holds or rings to allow easy use of the syringe. In such cases, it is beneficial to position the finger holds relative to each other to facilitate a user's hand movement. For example, as shown in FIG. 1B, once the first plunger 104 is extended, the finger holds 170 of the second plunger 107 are distal to the finger hold 110 of the first plunger 104. A user can place his thumb in the finger hold 110 of the first plunger and two other fingers in the finger holds 170 of the second plunger 107. To deliver fluid, the user uses his thumb to distally push the plunger 104 and the sealing body 114. The fingers in holds 170 provide leverage for the user as his thumb is pushing the first plunger 104. Upon delivering the fluid in the first barrel 101, the extended first plunger 104 is substantially inside the first barrel 101 (see FIG. 1C) and the finger hold 110 is proximal of the finger holds 170.

When fluid is collected, the user pulls on the finger holds 170 to proximally move the second plunger 170 partially out of the second barrel 102. When this occurs, the finger holds 170 may end up positioned proximal of the finger hold 110. To keep the finger holds 170 distal to the finger hold 110 (or return the finger holds 170 to a position distal of the finger hold 110); the user can shorten the length of the second plunger 107 by collapsing or retracting arms 109. As shown in FIG. 1C, the finger holds 170 of retracted second plunger 107 are distal of finger hold 110 of the extended first plunger 104. In some embodiments, the second plunger 107 retracts on itself when it is pushed in the distal direction, allowing it to shorten, and thus allowing the finger rings 170 to remain distal to the thumb in the finger ring 110 of the first plunger 104.

Following the collection step, in some embodiments, the syringe is sealed with a water tight cap. The capped syringe can be taken to the disposal receptacle with the contaminated fluid safely contained within, where it is discarded appropriately. In some embodiments, the syringe is a one-time use device that is disposable.

As can be appreciated, the irrigation and drainage syringe described may be made from any one or more of several materials such as plastic, glass, metal, rubber or any other suitable materials. Moreover, the syringe may come in many different sizes, providing different volumes of irrigation fluid for different procedural needs. Furthermore, the use of the terms irrigation, drainage, first, or second, are not intended to limit or narrow described embodiments to any particular order, sequence, or function. The embodiments described can be used for any number of procedures for moving and storing fluid or gas from and to target sites.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. An irrigation and drainage syringe comprising:
a first barrel;
a second barrel, wherein the second barrel is coaxial with the first barrel;
a nozzle positioned at a distal end of the syringe, wherein the nozzle comprises a first tip and a second tip, the first tip is positioned at least partially within the second tip;
a first passageway defined by the first barrel and first tip, wherein the first barrel is configured to store and move a first fluid in and out of the first passageway;
a second passageway defined by the second barrel and second tip, wherein the second barrel is configured to store and move a second fluid in and out of the second passageway, wherein the first fluid in the first passageway is separated from the second fluid in the second passageway and the first fluid does not contact the second passageway;
a first plunger connected to the first barrel; and
a second plunger connected to the second barrel, wherein the first plunger plunger can selectively move from a collapsed orientation to an extended orientation, the second plunger can selectively move from an extended orientation to a collapsed orientation, and the collapsed orientation having a shorter length than the extended orientation.

2. The syringe of claim 1, wherein the first barrel is pre-filled with a delivery fluid.

3. The syringe of claim 1, wherein the second barrel is configured to collect drainage fluid from a catheter in fluid communication with the second tip.

4. The syringe of claim 1, the second barrel comprising a distal wall, wherein the distal wall comprises at least one opening and the at least one opening is configured to allow fluid to enter and exit the second passageway.

5. The syringe of claim 1, wherein the first plunger and the second plunger comprise at least one or more finger rings on the proximal end of the syringe.

6. The syringe of claim 1, wherein the first plunger comprises an extendable arm, wherein the extendable arm comprises a plurality of segments configured to extend relative to one another when the first plunger is moved to the extended orientation and to retract into one another when the first plunger is moved to the collapsed orientation.

7. The syringe of claim 6, wherein the second plunger comprises:
a collapsible arm comprising a plurality of segments configured to retract relative to one another when the second plunger is pushed in a distal direction; and
a sealing body attached to the plunger, wherein the sealing body moves toward a proximal end of the syringe when the collapsible arm is pulled.

8. The syringe of claim 7 further comprising a latching mechanism on each of the segments of the first and second plunger arms, wherein the latching mechanism comprises a track on an inner surface of each segment and a flexible body attached to an outer surface of each of the plurality of segments, the flexible body configured to engage the track and preferentially allow movement of the segments in a direction that lengthens the arm of the first plunger and shortens the arm of the second plunger while resisting movement of the segments in an opposite direction.

9. The syringe of claim 1, wherein the second plunger comprises two finger holds and the first plunger comprises one finger hold positioned between the finger holds of the second plunger.

10. The syringe of claim 1 further comprising a locking mechanism, wherein the locking mechanism is configured to selectively lock either the first plunger or the second plunger and prevent movement of one plunger while the other plunger is in use, the locking mechanism comprising a mechanical connector for engaging a portion on either the first plunger or second plunger and to prevent movement of the engaged plunger.

11. The syringe of claim 1, wherein the nozzle comprises a convex protrusion configured to engage a catheter, the convex protrusion positioned proximal of the distal end of the nozzle.

12. The syringe of claim 1, wherein the nozzle comprises a groove along a circumference of the nozzle, the groove configured to engage a cap.

13. The syringe of claim 1 further comprising a splash guard.

14. The syringe of claim 1 further comprising a luer head adapter.

15. The syringe of claim 1 further comprising a syringe tip cap.

16. The syringe of claim 1 further comprising a luer head adapter and syringe tip cap assembly comprising:
a luer adapter;
a cap; and
a clamping mechanism comprising a compressible appendage biased toward a closed state, wherein compressing the appendage against the bias releases the clamp from the closed state.

17. Method for irrigating and draining a portion of a patient's body in need thereof comprising:
moving a first plunger of a multiple barrel syringe from a retracted state to an extended state by lengthening an arm of the first plunger;
locking the first plunger in the extended state;
depressing the first plunger to release fluid from a first barrel of the syringe into a portion of a patient's body, wherein the fluid passes through a first passageway; and
collecting fluid from the portion of the patient's body into a second barrel of the syringe, wherein the first barrel is coaxial with the second barrel and the collected fluid does not contact the first barrel and the first passageway.

18. The method of claim 17 further comprising collapsing a second plunger from an extended state comprising:
pulling an arm of the second plunger toward a proximal end of the syringe barrel; and applying a distally directed force to the arm.

19. The method of claim 18 further comprising locking the second plunger to prevent movement of the second plunger while the first plunger is in use.

20. The method of claim 18 further comprising locking the first plunger to prevent movement of the first plunger while the second plunger is in use.

21. The method of claim 17 further comprising connecting the syringe to a catheter in fluid communication with the portion of the patient's body.

22. A irrigation and drainage syringe comprising:
a proximal end and a distal end;
a first barrel extending along a longitudinal axis of the syringe, the first barrel configured to be coaxial with a second barrel, wherein the first barrel is partially positioned within the second barrel;
a nozzle positioned at the distal end of the syringe,
a first passageway defined by the first barrel;
a second passageway defined by the second barrel, wherein the second barrel can hold substantially the same or greater fluid volume as the first barrel;
a first plunger connected to the first barrel, wherein the first plunger is configured to move fluid into or out of the first passageway of the syringe;
a second plunger connected to the second barrel, wherein the second plunger is configured to move fluid into or out of the second passageway of the syringe, wherein fluid in the second passageway does not contact the first passageway;
a locking mechanism configured to selectively lock either the first plunger or the second plunger to prevent movement of the other plunger while one plunger is in use; and
a latching mechanism configured to releasably maintain a length of the first plunger, wherein the length of first plunger is adjustable.
